(19) United States
(12) Patent Application Publication
(10) Pub. No.: US 2017/0281379 A1
(43) Pub. Date: Oct. 5, 2017

(54) MECHANICALLY ASSISTED STENT DELIVERY SYSTEM

(71) Applicant: VENITI, INC., Fremont, CA (US)

(72) Inventors: Dan LAGOE, San Jose, CA (US); Stephen KAO, Mountain View, CA (US); Abha CHINUBHAI, Fremont, CA (US); Daniel RECINELLA, Queensbury, NY (US); Jeff ELKINS, Woodside, CA (US); Trudy NGUYEN, San Jose, CA (US)

(73) Assignee: VENITI, INC., Fremont, CA (US)

(21) Appl. No.: 15/472,990

(22) Filed: Mar. 29, 2017

Related U.S. Application Data
(60) Provisional application No. 62/314,804, filed on Mar. 29, 2016.
(60) Provisional application No. 62/334,010, filed on May 10, 2016.

(51) Int. Cl. A61F 2/95

(52) U.S. Cl. CPC A61F 2/95; A61F 2002/9505; A61F 2210/00

(57) ABSTRACT

A mechanical assisted delivery system and method are provided. One mechanical assisted delivery system includes an outer screw housing, a screw configured to rotate within the outer screw housing, an engagement mechanism configured for selective engagement with the screw, an outer shaft coupled to an end of the outer screw housing, a midshaft extending through the screw and a hub coupled to an end of the midshaft. Translational movement and rotational movement of the hub is configured to deploy a stent located within the outer shaft

### CROSS-REFERENCE TO RELATED APPLICATIONS

This nonprovisional application claims the benefit of and priority to U.S. Provisional Application No. 62/314,804, filed Mar. 29, 2016 and U.S. Provisional Application No. 62/334,010 filed May 10, 2016. The disclosures of the prior applications are hereby incorporated by reference herein in their entirety.

### BACKGROUND

Various embodiments relate generally to devices used for delivery of medical implants into hollow anatomical structures. More specifically, various embodiments relate to the delivery of stent implants through the use of a mechanically assisted delivery system.

The delivery of medical implants (including but not limited to stents) often requires use of a delivery system to constrain the implant and provide a means for low-profile advancement to the target location within the anatomy. One known delivery system construction achieves this by sheathing the implant inside a polymer tube prior to and during advancement to the desired deployment location. At the desired time and location of deployment, the polymer tube is withdrawn off of the implant allowing the implant to expand to its unconstrained geometry. This is commonly achieved using what is known as a “pin and push” or “pin and pull” technique in which the user constrains one end of the implant while pushing or pulling the polymer tube to expose and release the implant. Delivery systems of this design function by transferring force/motion directly from the user on the proximal (back) end of the device through the various shafts of the delivery system to actuate the unsheathing of the implant on the distal (front) end of the delivery system. Given the high radial strength and extreme compression of many such implants, the required delivery force can easily exceed that considered reasonable by Human Factors standards.

### SUMMARY

In one embodiment, a mechanical assisted delivery system is provided that includes an outer screw housing, a screw configured to rotate within the outer screw housing, an engagement mechanism configured for engagement with the screw, an outer shaft coupled to an end of the outer screw housing, a midshaft extending through the screw and a hub coupled to an end of the midshaft. Translational movement and rotational movement of the hub is configured to deploy a stent located within the outer shaft.

In another embodiment, a mechanical assisted delivery system is provided that includes an engagement mechanism having a screw and configured for engagement with a shaft, the shaft extending through the screw. The mechanical assisted delivery system further includes a hub coupled to an end of the shaft and configured to rotate, wherein rotational movement of the hub is configured to deploy a stent located at an end of the shaft.

In another embodiment, a method of deploying a stent is provided. The method includes deploying a stent using a screw type deployment mechanism actuated and caused to move rotationally or translationally by movement of a hub coupled to a shaft that extends within the screw type deployment mechanism.

### BRIEF DESCRIPTION OF THE DRAWINGS

FIGS. 1-3 are diagrams illustrating a mechanically assisted delivery system in accordance with an embodiment.

FIGS. 4-7 are diagrams illustrating a mechanically assisted delivery system in accordance with an embodiment in different deployed states.

FIGS. 8-11 are diagrams illustrating movement of a catheter assembly resulting from manipulation of handle components of the mechanically assisted delivery system illustrated in FIGS. 4-7.

FIGS. 12-17 are diagrams illustrating a mechanically assisted delivery system in accordance with another embodiment in different deployed states.

FIGS. 18-21 are diagrams illustrating movement of a catheter assembly resulting from manipulation of handle components of the mechanically assisted delivery system illustrated in FIGS. 12-17.

FIGS. 22 and 23 are diagrams illustrating a mechanically assisted delivery system in accordance with another embodiment.

FIG. 24 is a diagram illustrating a midshaft of the mechanically assisted delivery system illustrated in FIGS. 22 and 23.

FIGS. 25-28 are diagrams illustrating a mechanically assisted delivery system in accordance with an embodiment in different deployed states.

FIGS. 29-32 are diagrams illustrating movement of a catheter assembly resulting from manipulation of handle components of the mechanically assisted delivery system illustrated in FIGS. 24-27.

FIGS. 33-36 are diagrams illustrating a mechanically assisted delivery system in accordance with another embodiment in different deployed states.

FIGS. 37-40 are diagrams illustrating movement of a catheter assembly resulting from manipulation of handle components of the mechanically assisted delivery system illustrated in FIGS. 33-36.

### DETAILED DESCRIPTION OF EMBODIMENTS

The following detailed description of certain embodiments will be better understood when read in conjunction with the appended drawings. To the extent that the figures illustrate diagrams of the functional blocks of various embodiments, the functional blocks are not necessarily indicative of the division between various components. Thus, for example, one or more of the functional blocks may be implemented in a single piece of hardware or multiple pieces of hardware.

As used herein, the terms “system,” “subsystem,” “unit,” or “module” may include any combination of hardware that is operable or configured to perform one or more functions.

As used herein, an element or step recited in the singular and proceeded with the word “a” or “an” should be understood as not excluding plural of said elements or steps, unless such exclusion is explicitly stated. Furthermore, references to “one embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property.

Various embodiments provide systems and methods for stent delivery that include a design solution for overcoming high deployment force during deployment by the inclusion of a mechanically assisted handle into the design of the delivery system. In various embodiments, a handle replaces the “pin and push” or “pin and pull” designs that do not serve to provide mechanical advantage to the user. Mechanical assistance in some embodiments is accomplished by increasing or multiplying the user input force through mechanical means. Such force multiplication can be achieved using one or more mechanisms such as screws, gears, or pulleys, among other mechanisms. For example, in various embodiments, a threaded mechanism provides a mechanical advantage to the user during deployment operation. The threaded mechanism may form part of or be inserted within different types of stent deployment systems, such as a stent deployment system available from Veniti, to thereby deploy a stent, such as a stent available from Veniti.

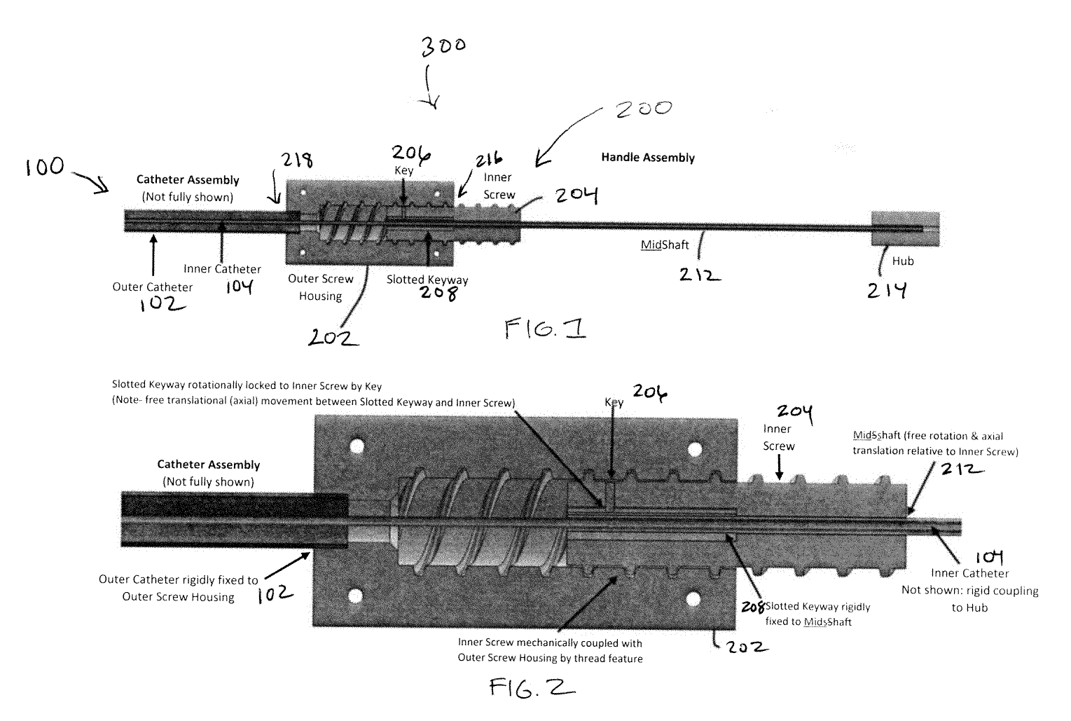

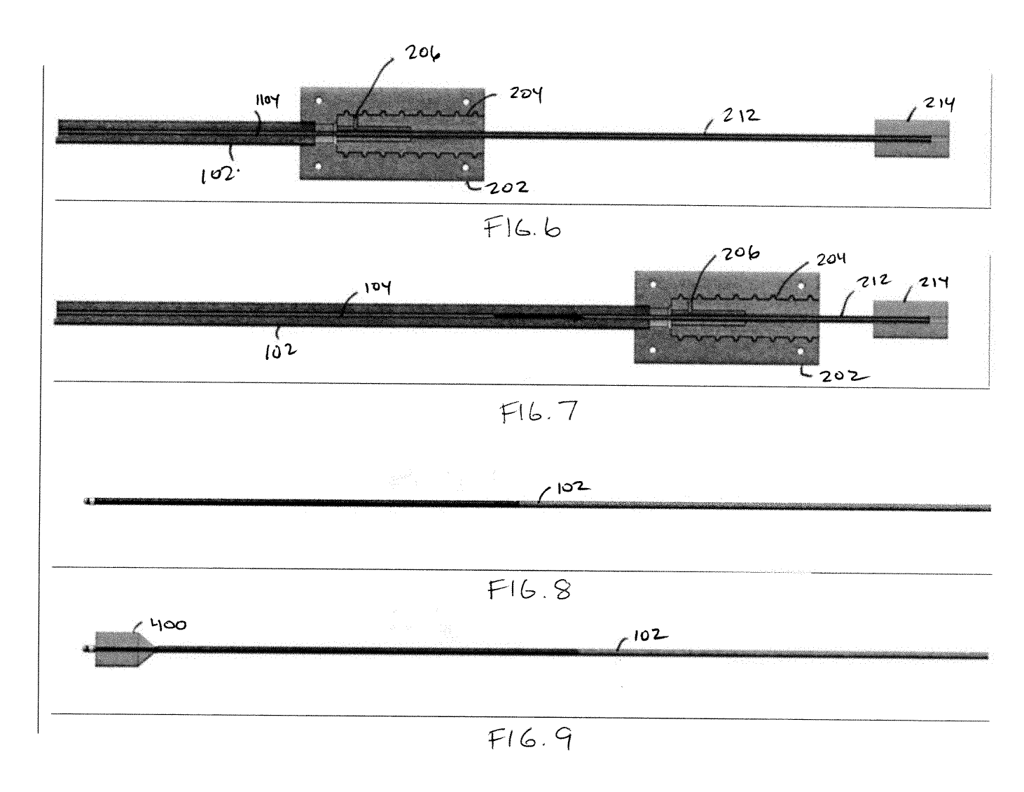

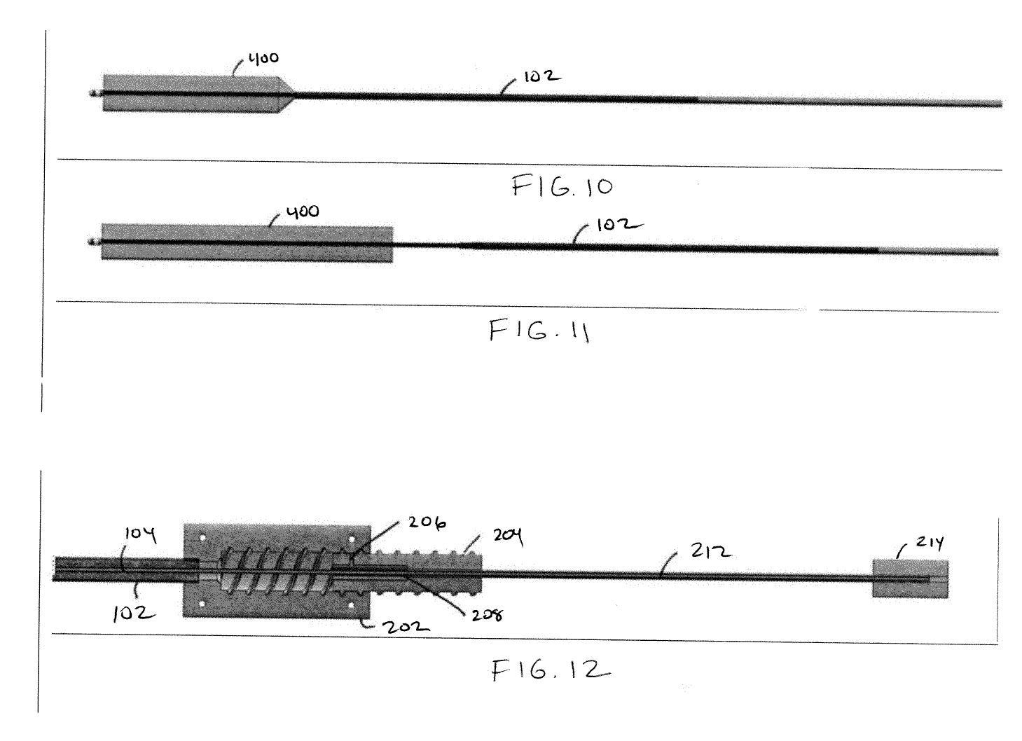

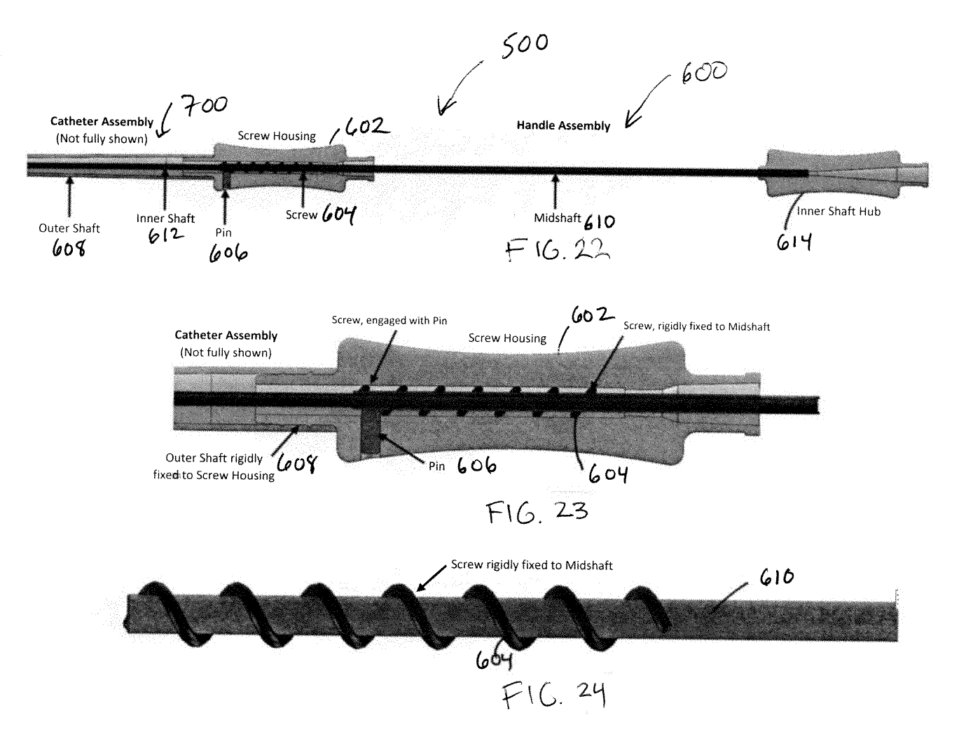

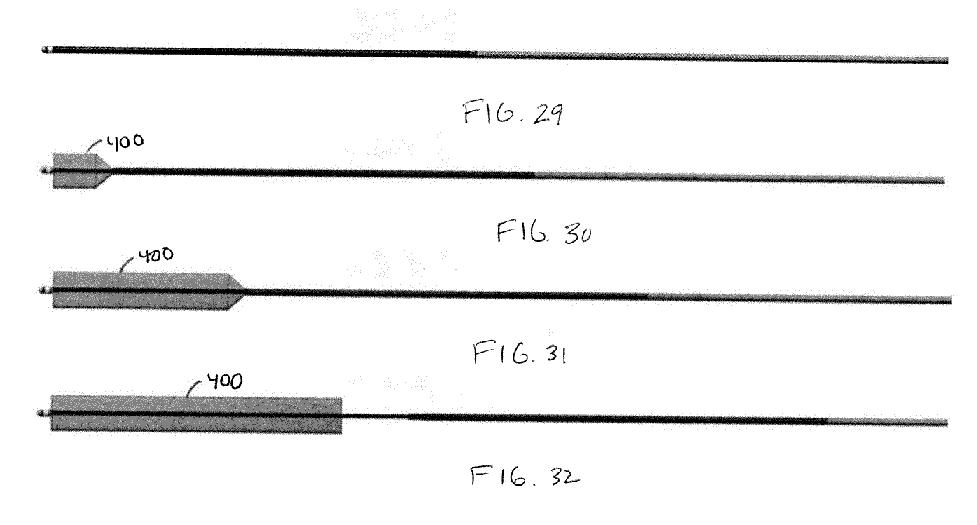

One or more embodiments as illustrated in FIGS. 1-40 include multiple components that can be grouped into two main sub-groups: the catheter assembly 100 and the handle assembly 200. The catheter assembly subgroup refers to (but is not limited to) a multitude of polymer shafts assembled in such a way as to constrain a stent implant and transmit force from the handle components to the implant during deployment. The handle assembly subgroup refers to (but is not limited to) a multitude of components that is manipulated by the user and transmits force to the various catheter components. These component operate together to provide the mechanically assisted stent delivery system 300.

The handle assembly 200 in various embodiments generally defines a delivery system handle that includes an outer screw housing 202, an inner screw 204, a key 206 and a slotted keyway 208 , an outer shaft 210, a midshaft 212, and a hub 214. The inner screw 204 is housed, at least partially inside the outer screw housing 202, and is mechanically coupled through a mating thread feature of the components. The complementary threaded arrangement may be varied as desired or needed, such as the size and pitch of the threads to define different mechanical advantages.

The slotted keyway 208 is rigidly attached to one end of the midshaft 212 (as seen more clearly in FIG. 3) and positioned in the inner diameter of the inner screw 204 (e.g., extending longitudinally within the inner screw 204). The key 206 is embedded in the sidewall of the inner screw 204 and is configured to rotationally lock the slotted keyway 208 to the inner screw 204 while still allowing translational movement along the longitudinal axis. It should be noted that the key 206 may be embedded at any portion along the length of the inner screw 204 and the position illustrated in the figures is merely for example.

The hub 214 is rigidly attached to an opposing end of the midshaft 214 and provides an enlarged gripping surface for rotational/translational input from the user. An outer shaft, illustrated as outer catheter 102 is rigidly attached to an end 218 of the outer screw housing 202 opposite to an end to which the midshaft 212 is coupled, and rotates when the outer screw housing rotates 202. It should be appreciated that that an inner catheter 104 extends within the outer catheter 204. Thus, as can be seen more particularly in FIGS. 1-3 that illustrate an embodiment of the handle assembly construction, the slotted keyway 208 is rotationally locked to the inner screw 204 by the key 206. It should be noted that in the illustrated configuration, free translation (axial) movement is provided between the slotted keyway 208 and the inner screw 204.

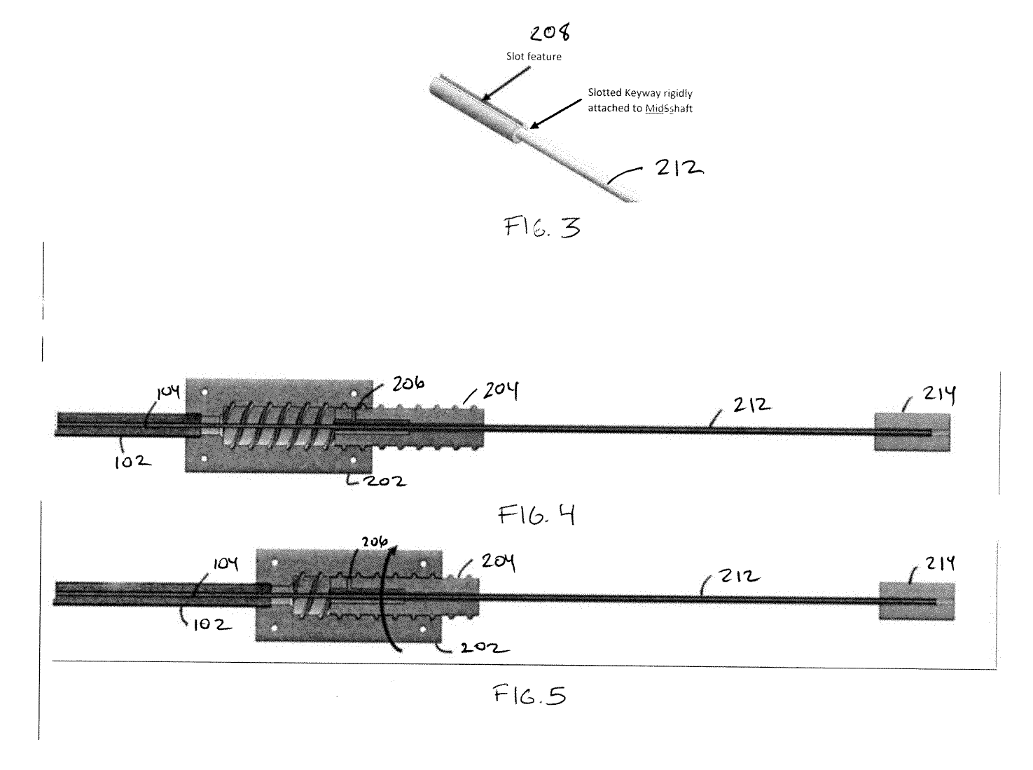

In operation, and with reference particularly to FIGS. 4-7, the mechanically assisted stent delivery system 300 accommodates the user of the device to “pin and pull” to deploy the stent. The midshaft 212 and hub 214 are considered fixed relative to the rest of the delivery system and these parts are held “pinned”. The outer catheter 102 can then be “pulled” relative to the midshaft 212 and hub 214 such that the force transmitted to the stent, which is also stationary, enables the stent to slidably move and deploy out of the outer catheter 102. Alternatively, because the outer catheter 102 is coupled to the outer screw housing 202, rotating the outer catheter 102 or the outer screw housing 202 causes these components to move relative to the midshaft 212 and hub 214, and enables the stent to slidably move and deploy out of the outer catheter 102.

In one deployment method, where the outer catheter 102 and the outer screw housing 202 are moved translationally, deployment of the stent can be done quickly with less control by the user. In a second deployment method where the outer catheter 102 and the outer screw housing 202 are moved rotationally, deployment of the stent can be done slowly with more control by the user. If the stent deployment requires a high force, the user may use the second deployment method to initiate stent deployment, and once the initial high deployment force is broken, then the user may pull back using the first deployment method to fully deploy the stent.

FIGS. 4-11 illustrate hand and delivery system functionality in accordance with various embodiments. More particularly, FIG. 4 illustrates a 0% deployed position with no handle actuation. FIG. 5 illustrates a partial deployed position (less than 20%) wherein the inner screw 204 has been rotated (in this embodiment, clockwise rotation) to cause movement in a deployment direction such that the inner screw 204 moves left as viewed in the figure. Thus, FIG. 5 illustrates some rotation of the inner screw 204. As can be seen, more of the length of the inner screw 204 is moved within the outer screw housing 202 and the midshaft 212 is also caused to be moved to deploy a stent as shown in FIGS. 8-11. FIGS. 6 and 7 show the result of additional rotation of the inner screw 204 relative to the outer screw housing 202 by manipulation of the handle components by a user. As can be seen, the inner screw 204 rotates to be entirely within the outer screw housing 202 in this embodiment. FIG. 7 illustrates a 100% deployed position. Thus, as illustrated in FIGS. 8-11, which correspond to the stent position corresponding to the deployed positions of FIGS. 4-7, a stent 400 is caused to be deployed (FIG. 11 illustrating a fully deployed stent 400), expanded in this example, by the manipulation of the handle components as described above. In the above described embodiment, an engagement mechanism is defined that is configured for selective engagement.

In another deployment configuration, and with particular reference to FIGS. 12-21, the mechanically assisted stent delivery system 300 accommodates the user to “pin and push” to deploy the stent 400. In this embodiment of the assembled handle configuration, the outer screw housing 202 is considered fixed. The outer catheter 102 is rigidly attached to the outer screw housing 202. In operation, rotation of the hub 214 transmits force through the midshaft 212, which is then transmitted to the inner screw 204. Rotation of the inner screw 204 results in axial (translational) movement of all handle components relative to the fixed outer screw housing 202. The inner catheter 104 is coupled to the hub 214 such that the force and axial movement is transferred through the catheter assembly to initiate deployment of the implant (stent 400) on the distal (front) end of the delivery system. Once deployment has been initiated, breaking the initial high deployment force, the user may press forward on the hub 214 to fully deploy the implant (stent).

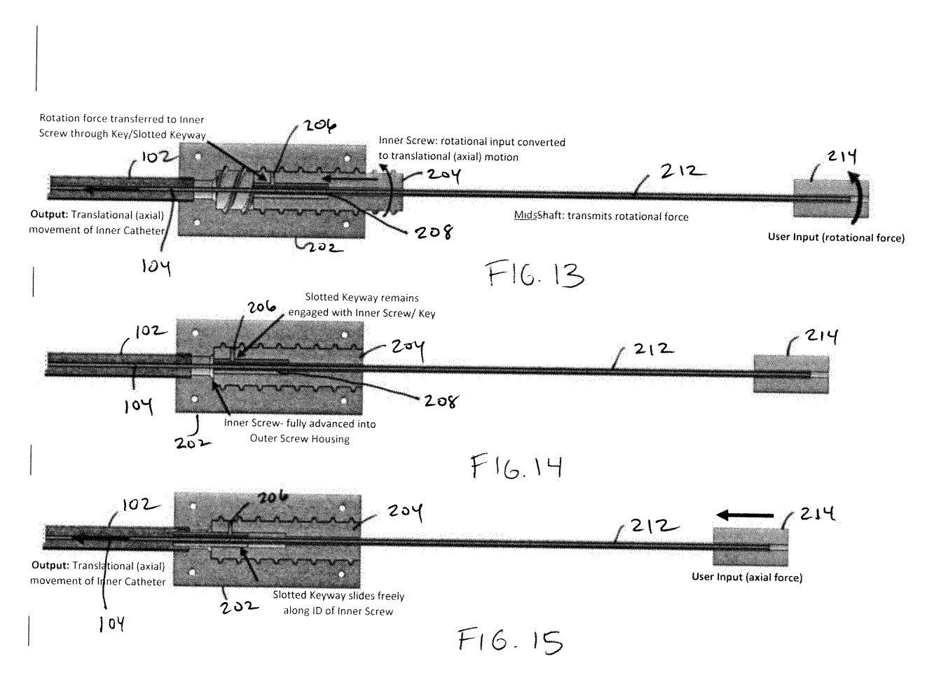

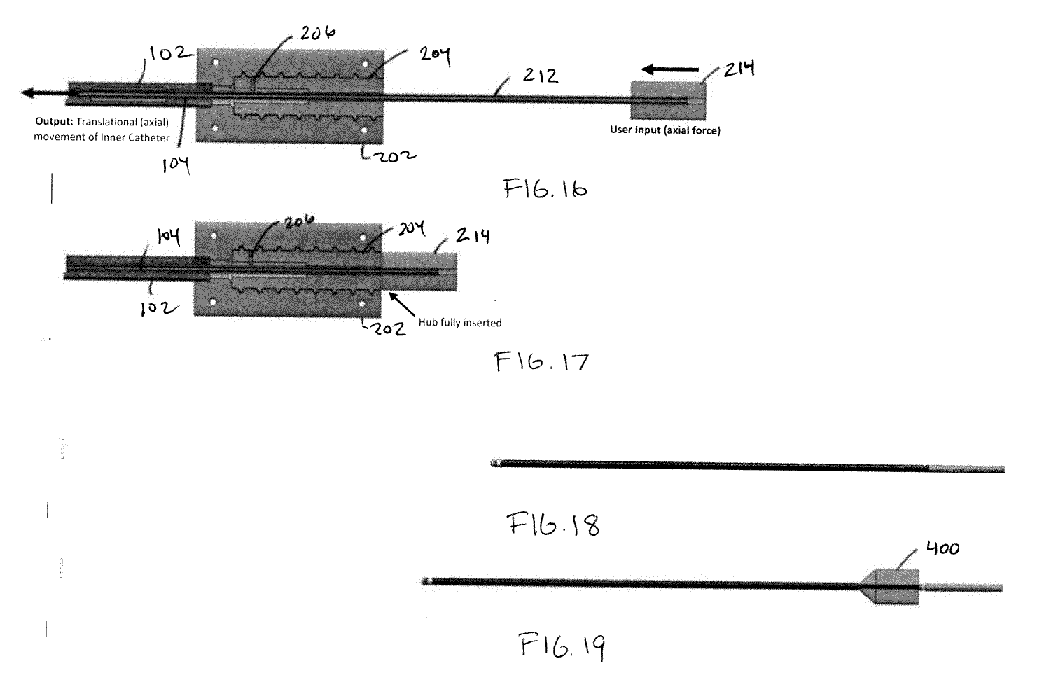

More particularly, FIG. 12 illustrates a 0% deployed position, FIGS. 13 and 14 illustrate a less than 20% deployed position, FIG. 15 illustrates a less than 50% deployed position, FIG. 16 illustrates a 50% deployed position and FIG. 17 illustrates a 100% deployed position. The relative position of the catheter and stent 400 are shown in FIGS. 18-21, corresponding to 0%, 20%, 50% and 100% deployed positions. In this operational embodiment, rotation input, which is rotational movement of the inner screw 204 is converted to translational (axial) movement. In particular, a user manipulation of the hub 214, illustrated as rotational force of the hub 214, is transmitted to the inner screw 204 by the midshaft 212. Specifically, rotational force is transmitted to the inner screw 204 through the key 206 and slotted keyway 208. As a result, an output translation (axial) force causes movement of the inner catheter 104. As can be seen in FIG. 14, the inner screw 204 is fully advanced into the outer screw housing 202 and the slotted keyway 208 remains engaged with the inner screw 204 and key 206. However, in FIG. 14, the slotted keyway 208 slides freely along the inner diameter of the inner screw 204. Moreover, as can be seen in FIG. 17, the hub 214 abuts against the end 216 of the outer screw housing 202. In the above described embodiment, an engagement mechanism is defined that is configured for selective engagement.

Variations and modifications are contemplated. For example, the slotted keyway may be replaced by a geometry that provides equivalent functionality:

(i) Non-circular midshaft (e.g., ovalized, ‘D’ shaped, square, hexagonal, etc.) with corresponding geometry on inner screw through hole.

(ii) Non-circular feature on distal midshaft which mates with corresponding geometry on inner screw.

In some embodiments, the handle may be designed such that the outer screw housing 202 is rotated and the hub 214 remains fixed during use. Also, component geometry such as length, diameter, and travel distance (among other geometry components) may be modified while maintaining the described functionality. Additionally, the pitch of the screw mechanism may be adjusted (higher or lower) to obtain a desired travel per revolution. In some embodiments, a desirable pitch is from 1 revolution per inch to 8 revolutions per inch. However, other pitches may be used as desired or needed.

It should be noted that while the various embodiments of a mechanical assisted handle mechanism described herein are designed for use with stent delivery systems, one or more embodiments may be applied to or used in other medical implant delivery systems, such as systems that have similar deployment methods.

Thus, one or more embodiments provide a mechanical assisted delivery system 300 that includes an outer screw housing, an inner screw, a key and slotted keyway, an outer shaft, a midshaft, and a hub. In various embodiments, the mechanical assisted delivery system 300 deploys a stent through both translational movement and rotational movement of the handle mechanism. In some embodiments, the midshaft may be comprised of metal hypotubing or high compression strength plastic tubing (e.g., PEEK shaft) and the pitch of screw mechanism (outer screw housing and inner screw) is between 1 revolution per inch to 8 revolutions per inch.

In some embodiments, the outer screw housing 202 and inner screw 204 move rotationally relative to each other when turned. In other embodiments, the outer screw housing 202 and inner screw 204 engage and stay fixed relative to each other, and move together as a unit when pushed or pulled. The midshaft 212 with slotted keyway 208 and inner screw 204 move slidably and translationally relative to each other in some embodiments. In other embodiments, the midshaft 212 with slotted keyway 208 and inner screw 204 engage, stay fixed relative to each other, and rotate together as a unit when turned.

In operation in various embodiments, rotation of the outer screw housing 202 relative to the inner screw 204 deploys the stent 400. In various embodiments, translation of the midshaft 212 relative to the outer screw housing 202 with outer shaft deploys the stent 400.

Various embodiments provide torsion of the inner screw 204 that provides mechanical advantage and initiates deployment of the implant. Additionally, the design of various embodiments allows for either rotational or translational user input on the hub 214 to actuate deployment without engagement/disengagement of screw mechanism.

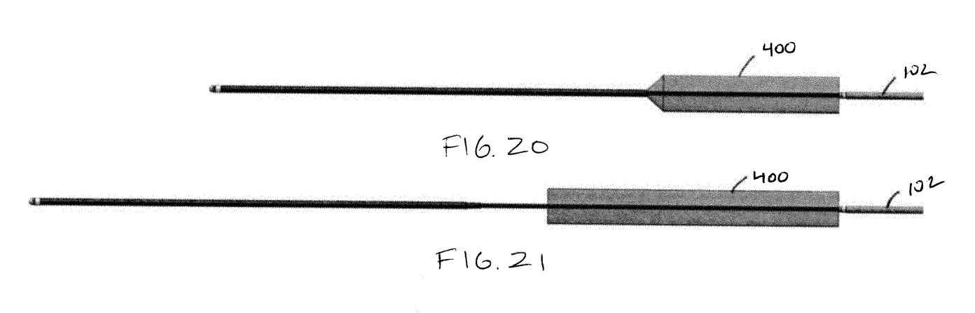

Variations and modifications are contemplated. For example, FIGS. 21-39 illustrate another embodiment of a mechanically assisted stent delivery system 500. In this embodiment, the mechanically assisted stent delivery system 500 includes multiple components that can be grouped into two main sub-groups: the catheter assembly 700 and the handle assembly 600. The catheter assembly subgroup refers to (but is not limited to) a multitude of polymer shafts assembled in such a way as to constrain a stent implant and transmit force from the handle components to the implant during deployment. The handle assembly subgroup refers to (but is not limited to) a multitude of components that is manipulated by the user and transmits force to the various catheter components. These component operate together to provide the mechanically assisted stent delivery system 500. In the below described embodiments, an engagement mechanism is defined that is configured for permanent engagement or non-selective engagement.

The handle assembly 600 that defines a delivery system handle includes a screw housing 602, a screw 604, a pin 606, an outer shaft 608, a midshaft 610, an inner shaft 612, and inner shaft hub 614. In this embodiment, the screw 604 is mechanically coupled to the screw housing 602 through engagement with the pin 606, which is embedded in the sidewall of the screw housing 602. The screw 604 and screw housing 602 may move rotationally relative to one another resulting in relative translational movement between the components due to the interaction between the screw 604 and pin 606. The screw 604 is rigidly attached to the midshaft 610. For example, in some embodiments, the screw 604 is wound around and coupled to an outer surface of the midshaft 610. The inner shaft hub 614 is rigidly attached to the opposing end of the midshaft 610 and provides an enlarged gripping surface for the user. The outer shaft 608 (outer catheter) is rigidly attached to the end of the screw housing 602 (opposite to the end at which the inner shaft hub 614 is located). The pin 606 may be coupled with the screw 604.

In the illustrated embodiment, the mechanically assisted stent delivery system 500 accommodates the use of an enhanced “pin and pull” method to deploy the stent 400 as shown more particularly in FIGS. 25-32. The midshaft 610 and inner shaft hub 614 are considered fixed relative to the rest of the delivery system and are “pinned” by the user. Since the outer shaft 608 is coupled to the screw housing 602, and the screw housing 602 is mechanically coupled to the screw 604 through engagement with the pin 606, rotating the outer shaft 608 or the screw housing 602 results in translational movement of the outer shaft 608 and screw housing 602 relative to the midshaft 610 and inner shaft hub 614. The resulting translational movement of the outer shaft 608 and screw housing 608 enables the stent 400 to slidably move and deploy out of the outer shaft 608. Once the screw housing 602 has been fully rotated and advanced over the screw 504, the user may slidably move the screw housing 602 and outer shaft 608 to fully deploy the stent out of the outer shaft 608.

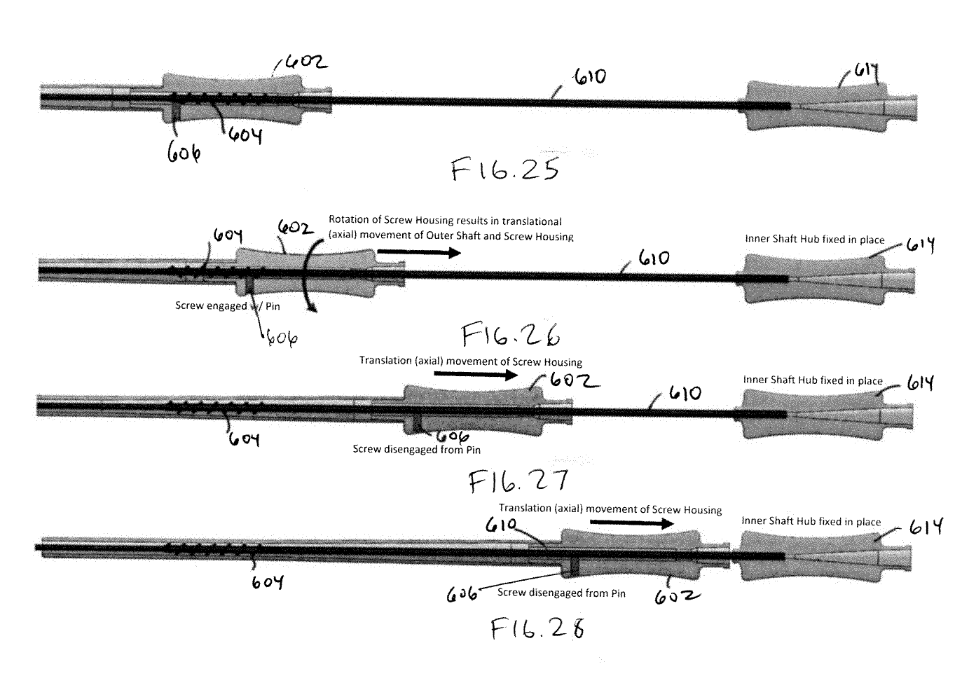

More particularly, FIG. 25 illustrates a 0% deployed position, FIG. 26 illustrates a 20% deployed position, FIG. 27 illustrates a 50% deployed position and FIG. 28 illustrates a 100% deployed position. The relative position of the catheter and stent 400 are shown in FIGS. 29-32, corresponding to 0%, 20%, 50% and 100% deployed positions. In this operational embodiment, rotation input, which is rotational movement of the screw housing 602 results in translation movement of the outer shaft 608 and screw housing 602. With the screw 604 engaged with the pin 606, this rotational movement causes translational movement (see FIG. 26). With the screw 604 disengaged from the pin 606 (see FIG. 27 translation (axial) movement of the screw housing 602 is provided (see FIGS. 27 and 28). It should be noted that engagement and disengagement in the various embodiments may be provided manually or automatically, such as when reaching a defined translational position.

In another operational configuration (see FIGS. 33-40), the mechanically assisted stent delivery system 500 accommodates the use of an enhanced “pin and push” method to deploy the stent 400. In this embodiment, the screw housing 602 and outer shaft 608 are considered fixed and are “pinned” by the user. Rotation of the inner shaft hub 614 transmits force through the midshaft 610, which is then transmitted to the screw 604. Rotation of the screw 604 results in translational (axial) movement of the inner shaft 612, midshaft 610, and inner shaft hub 614 relative to the fixed screw housing 602 and outer shaft 608. The resulting translational (axial) movement of the inner shaft 612 enables the stent 400 to slidably move and deploy out of the outer shaft 608. Once deployment has been initiated, breaking the initial high deployment force, the user may press forward on the inner shaft hub 614 to fully deploy the implant (stent 400).

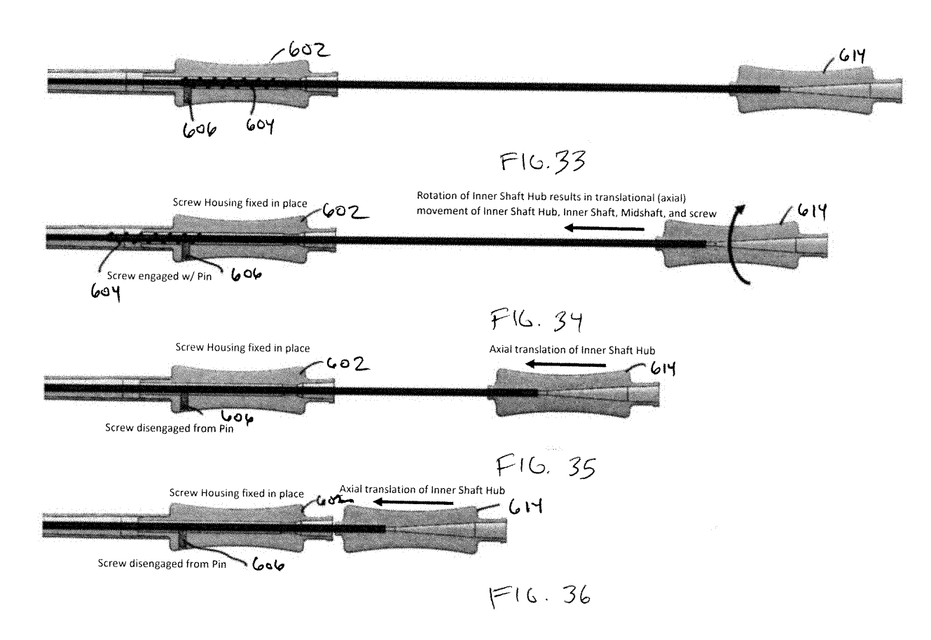

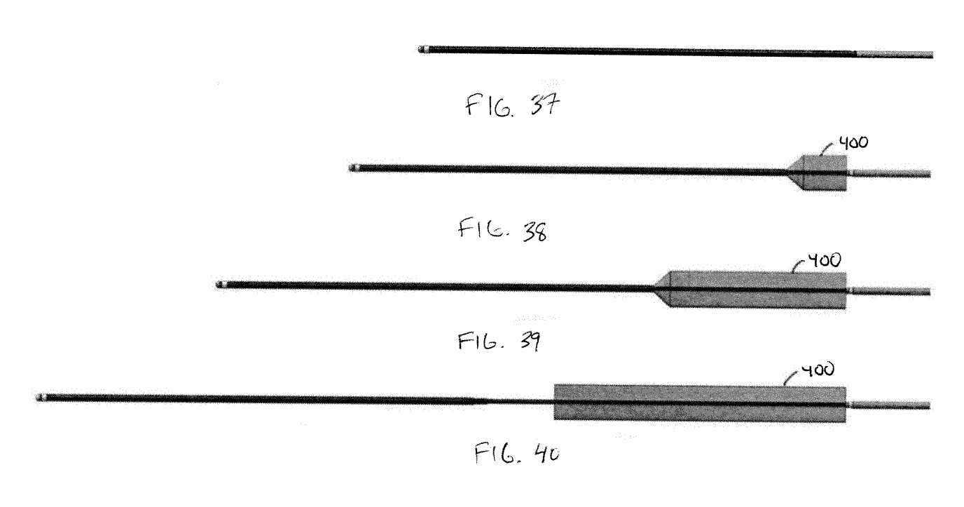

More particularly, FIG. 33 illustrates a 0% deployed position, FIG. 34 illustrates a 20% deployed position, FIG. 35 illustrates a 50% deployed position and FIG. 36 illustrates a 100% deployed position. The relative position of the catheter and stent 400 are shown in FIGS. 37-40, corresponding to 0%, 20%, 50% and 100% deployed positions. In this operational embodiment, rotation input, which is rotational movement of the inner shaft hub 614 results in translational (axial) movement of the inner shaft hub 614, the inner shaft 612, the midshaft 610 and the screw 604. This movement occurs when the screw 604 is engaged with the pin 606 (see FIG. 34). With the screw 604 disengaged from the pin 606, translational (axial) movement of the inner shaft hub 614 is provided (see FIG. 35). It should be noted that the screw housing 602 is fixed in place.

Modifications to these embodiments are contemplated. For example, the pin 606 (illustrated in FIGS. 22-28) may be replaced with a selectively engageable/disengageable component (e.g., adjustable screw, toggle switch, push button, or equivalent). Use of a selectively engageable component allows the user to engage or disengage the screw housing 602 from the screw 604. In doing so, the stent 400 may be deployed through either rotational input to the screw housing 602 or outer shaft 608 when engaged, or translational (axial) input to the screw housing 602 or outer shaft 608 when the disengaged.

In another embodiment, the pin 606 (illustrated in FIGS. 33-36) is replaced with a selectively engageable/disengageable component (e.g., adjustable screw, toggle switch, push button, or equivalent). Use of a selectively engageable component allows the user to engage or disengage the screw housing 602 from the screw 604. In doing so, the stent 400 may be deployed through either rotational input to the inner shaft hub 614 when engaged, or translational (axial) input to the inner shaft hub 614 when disengaged.

In these embodiments, torsion of the screw 604 provides mechanical advantage and initiates deployment of the implant. Engagement with the screw feature may be selectable. In some variations, the handle may be designed such that the screw housing 602 is rotated and the inner shaft hub 214 remains fixed during use. Additionally, the component geometry such as length, diameter, and travel distance may be modified while maintaining the herein described functionality. The mechanically assisted stent delivery system is configured for use with stent deployment, but may be applied to other medical implant delivery systems that have similar deployment methods. The pitch of the screw 604 may be adjusted (higher or lower) to obtain the desired travel per revolution. A desirable pitch is from 1 revolution per inch to 8 revolutions per inch. The pin 606 may either be fixed or selectively engaged/disengaged from the screw 604 to facilitate different deployment techniques.

This written description uses examples to disclose the various embodiments, including the best mode, and also to enable any person skilled in the art to practice the various embodiments, including making and using any devices or systems and performing any incorporated methods. The patentable scope of the various embodiments is defined by the claims, and may include other examples that occur to those skilled in the art. Such other examples are intended to be within the scope of the claims if the examples have structural elements that do not differ from the literal language of the claims, or if the examples include equivalent structural elements with insubstantial differences from the literal languages of the claims.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. Dimensions, types of materials, orientations of the various components, and the number and positions of the various components described herein are intended to define parameters of certain embodiments, and are by no means limiting and are merely exemplary embodiments. Many other embodiments and modifications within the spirit and scope of the claims will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. §112(f) paragraph, unless and until such claim limitations expressly use the phrase “means for” followed by a statement of function void of further structure.

What is claimed is:

1. A mechanical assisted delivery system comprising:
an outer screw housing;
a screw configured to rotate within the outer screw housing;
an engagement mechanism configured for engagement with the screw;
an outer shaft coupled to an end of the outer screw housing;
a midshaft extending through the screw; and
a hub coupled to an end of the midshaft,
wherein, translational movement and rotational movement of the hub is configured to deploy a stent located within the outer shaft.

2. The mechanical assisted delivery system of claim 1, wherein the engagement mechanism comprises a key and a slotted keyway, wherein the slotted keyway is coupled to the midshaft, wherein in a disengaged state, the slotted keyway slides along an inner diameter of the screw.

3. The mechanical assisted delivery system of claim 1, wherein the engagement mechanism comprises a pin coupled to the screw.

4. The mechanical assisted delivery system of claim 3, wherein the screw is wound around the midshaft.

5. The mechanical assisted delivery system of claim 1, wherein the midshaft comprises at least one of metal hypotubing or high compression strength plastic tubing.

6. The mechanical assisted delivery system of claim 1, wherein a pitch of the screw defined by the outer screw housing and the screw is between 1 revolution per inch to 8 revolutions per inch.

7. The mechanical assisted delivery system of claim 1, wherein the outer screw housing and the screw are configured to move rotationally relative to each other when turned.

8. The mechanical assisted delivery system of claim 1, wherein the outer screw housing and the screw are configured to engage, stay fixed relative to each other, and move together as a unit when pushed or pulled.

9. The mechanical assisted delivery system of claim 1, wherein the engagement mechanism comprises a key and a slotted keyway and the midshaft with slotted keyway and screw are configured to move slidably and translationally relative to each other.

10. The mechanical assisted delivery system of claim 1, wherein the engagement mechanism comprises a key and a slotted keyway and the midshaft with slotted keyway and inner screw are configured to engage, stay fixed relative to each other, and rotate together as a unit when turned.

11. The mechanical assisted delivery system of claim 1, wherein rotation of the outer screw housing relative to the screw deploys the stent.

12. The mechanical assisted delivery system of claim 1, wherein translation of the midshaft relative to the outer screw housing with outer shaft deploys the stent.

13. The mechanical assisted delivery system of claim 1, wherein the engagement mechanism comprises a pin coupled to screw and the outer screw housing and the screw are configured to move rotationally and translationally relative to each other.

14. The mechanical assisted delivery system of claim 1, wherein the engagement mechanism comprises a pin coupled to the screw and the midshaft and screw are configured to engage, stay fixed relative to each other, and rotate together as a unit when turned.

15. The mechanical assisted delivery system of claim 1, wherein the engagement mechanism is configured for selective engagement.

16. The mechanical assisted delivery system of claim 1, wherein the engagement mechanism is configured for permanent engagement.

17. A mechanical assisted delivery system comprising:
an engagement mechanism having a screw and configured for engagement with a shaft, the shaft extending through the screw; and
a hub coupled to an end of the shaft and configured to rotate,
wherein rotational movement of the hub is configured to deploy a stent located at an end of the shaft.

18. The mechanical assisted delivery system of claim 17, wherein the hub is configured to translate.

19. The mechanical assisted delivery system of claim 18, wherein the hub is configured to rotate during initial deployment of the stent and configured to translate after the initial deployment.

20. A method of deploying a stent, the method comprising:
deploying a stent using a screw type deployment mechanism actuated and caused to move rotationally or translationally by movement of a hub coupled to a shaft that extends within the screw type deployment mechanism.
